SEQUENCE LISTING (3 sequences)

SEQ ID NO: 1 - RNA, 1299 nt, Homo sapiens
auguugggcaacucugcgccggggccugcgacccgcgaggcgggcucggcgcugcuagcauugcagcagacggcgcuccaagaggaccaggagaauaucaacccggaaaaggcagcgcccguccaacaaccgcggacccgggccgcgcuggcgguacugaaguccgggaacccgcggggucuagcgcagcagcagaggccgaagacgagacggguugcaccccuuaaggaucuuccuguaaaugaugagcaugucaccguuccuccuuggaaagcaaacaguaaacagccugcguucaccauucauguggauccagcagaaaaagaagcucagaagaagccagcugaaucucaaaaaauagagcgugaagaugcccuggcuuuuaauucagccauuaguuuaccuggacccagaaaaccauuggucccucuugauuauccaauggaugguaguuuugagucaccacauacuauggacaugucaauuguauuagaagaugaaaagccagugaguguuaaugaaguaccagacuaccaugaggauauucacacauaccuuagggaaauggagguuaaauguaaaccuaaaguggguuacaugaagaaacagccagacaucacuaacaguaugagagcuauccucguggacugguuaguugaaguaggagaagaauauaaacuacagaaugagacccugcauuuggcugugaacuacauugauagguuccugucuuccaugucagugcugagaggaaaacuucagcuugugggcacugcugcuaugcuguuagccucaaaguuugaagaaauauaccccccagaaguagcagaguuuguguacauuacagaugauaccuacaccaagaaacaaguucugagaauggagcaucuaguuuugaaaguccuuacuuuugacuuagcugcuccaacaguaaaucaguuucuuacccaauacuuucugcaucagcagccugcaaacugcaaaguugaaaguuuagcaauguuuuugggagaauuaaguuugauagaugcugacccauaccucaaguauuugccaucaguuauugcuggauccgccuuucauuuagcacucuacacagucacgggacaaagcuggccugaaucauuaauacgaaagacuggauauacccuggaaagucuuaagccuugucucauggaccuucaccagaccuaccucaaagcaccacagcaugcacaacagucaauaagagaaaaguacaaaaauucaaaguaucaugguguuucucuccucaacccaccagagacacuaaaucuguaa

SEQ ID NO: 2 - RNA, 1197 nt, Homo sapiens
auggcgcugcuccgacgcccgacgguguccagugauuuggagaauauugacacaggaguuaauucuaaaguuaagagucaugugacuauuaggcgaacuguuuuagaagaaauuggaaauagaguuacaaccagagcagcacaaguagcuaagaaagcucagaacaccaaaguuccaguucaacccaccaaaacaacaaaugucaacaaacaacugaaaccuacugcuucugucaaaccaguacagauggaaaaguuggcuccaaaggguccuucucccacaccugaggaugucuccaugaaggaagagaaucucugccaagcuuuuucugaugccuugcucugcaaaaucgaggacauugauaacgaagauugggagaacccucagcucugcagugacuacguuaaggauaucuaucaguaucucaggcagcuggagguuuugcaguccauaaacccacauuucuuagauggaagagauauaaauggacgcaugcgugccauccuaguggauccgcugguacaaguccacuccaaguuuaggcuucugcaggagacucuguacaugugcguuggcauuauggaucgauuuuuacagguucagccaguuucccggaagaagcuucaauuaguugggauuacugcucugcucuuggcuuccaaguaugaggagauguuuucuccaaauauugaagacuuuguuuacaucacagacaaugcuuauaccaguucccaaauccgagaaauggaaacucuaauuuugaaagaauugaaauuugaguugggucgacccuugccacuacacuucuuaaggcgagcaucaaaagccggggagguugauguugaacagcacacuuuagccaaguauuugauggagcugacucucaucgacuaugauauggugcauuaucauccuucuaagguagcagcagcugcuuccugcuugucucagaaggauccaggacaaggaaaauggaacuuaaagcagcaguauuacacaggauacacagagaaugaaguauuggaagucaugcagcacauggccaagaauguggugaaaguaaaugaaaacuuaacuaaauucaucgccaucaagaauaaguaugcaagcagcaaacuccugaagaucagcaugaucccucagcugaacucaaaagccgucaaagaccuugccuccccacugauaggaagguccuag

SEQ ID NO: 3 - DNA, 10610 bp, Artificial Sequence
  Description of Artificial Sequence Synthetic vector sequence
gatggcggatgtgtgacatacacgacgccaaaagattttgttccagctcctgccacctccgctacgcgagagattaaccacccacgatggccgccaaagtgcatgttgatattgaggctgacagcccattcatcaagtctttgcagaaggcatttccgtcgttcgaggtggagtcattgcaggtcacaccaaatgaccatgcaaatgccagagcattttcgcacctggctaccaaattgatcgagcaggagactgacaaagacacactcatcttggatatcggcagtgcgccttccaggagaatgatgtctacgcacaaataccactgcgtatgccctatgcgcagcgcagaagaccccgaaaggctcgatagctacgcaaagaaactggcagcggcctccgggaaggtgctggatagagagatcgcaggaaaaatcaccgacctgcagaccgtcatggctacgccagacgctgaatctcctaccttttgcctgcatacagacgtcacgtgtcgtacggcagccgaagtggccgtataccaggacgtgtatgctgtacatgcaccaacatcgctgtaccatcaggcgatgaaaggtgtcagaacggcgtattggattgggtttgacaccaccccgtttatgtttgacgcgctagcaggcgcgtatccaacctacgccacaaactgggccgacgagcaggtgttacaggccaggaacataggactgtgtgcagcatccttgactgagggaagactcggcaaactgtccattctccgcaagaagcaattgaaaccttgcgacacagtcatgttctcggtaggatctacattgtacactgagagcagaaagctactgaggagctggcacttaccctccgtattccacctgaaaggtaaacaatcctttacctgtaggtgcgataccatcgtatcatgtgaagggtacgtagttaagaaaatcactatgtgccccggcctgtacggtaaaacggtagggtacgccgtgacgtatcacgcggagggattcctagtgtgcaagaccacagacactgtcaaaggagaaagagtctcattccctgtatgcacctacgtcccctcaaccatctgtgatcaaatgactggcatactagcgaccgacgtcacaccggaggacgcacagaagttgttagtgggattgaatcagaggatagttgtgaacggaagaacacagcgaaacactaacacgatgaagaactatctgcttccgattgtggccgtcgcatttagcaagtgggcgagggaatacaaggcagaccttgatgatgaaaaacctctgggtgtccgagagaggtcacttacttgctgctgcttgtgggcatttaaaacgaggaagatgcacaccatgtacaagaaaccagacacccagacaatagtgaaggtgccttcagagtttaactcgttcgtcatcccgagcctatggtctacaggcctcgcaatcccagtcagatcacgcattaagatgcttttggccaagaagaccaagcgagagttaatacctgttctcgacgcgtcgtcagccagggatgctgaacaagaggagaaggagaggttggaggccgagctgactagagaagccttaccacccctcgtccccatcgcgccggcggagacgggagtcgtcgacgtcgacgttgaagaactagagtatcacgcaggtgcaggggtcgtggaaacacctcgcagcgcgttgaaagtcaccgcacagccgaacgacgtactactaggaaattacgtagttctgtccccgcagaccgtgctcaagagctccaagttggcccccgtgcaccctctagcagagcaggtgaaaataataacacataacgggagggccggcggttaccaggtcgacggatatgacggcagggtcctactaccatgtggatcggccattccggtccctgagtttcaggctttgagcgagagcgccactatggtgtacaacgaaagggagttcgtcaacaggaaactataccatattgccgttcacggaccctcgctgaacaccgacgaggagaactacgagaaagtcagagctgaaagaactgacgccgagtacgtgttcgacgtagataaaaaatgctgcgtcaagagagaggaagcgtcgggtttggtgttggtgggagagctaaccaaccccccgttccatgaattcgcctacgaagggctgaagatcaggccgtcggcaccatataagactacagtagtaggagtctttggggttccgggatcaggcaagtctgctattattaagagcctcgtgaccaaacacgatctggtcaccagcggcaagaaggagaactgccaggaaatagttaacgacgtgaagaagcaccgcgggaaggggacaagtagggaaaacagtgactccatcctgctaaacgggtgtcgtcgtgccgtggacatcctatatgtggacgaggctttcgcttgccatcccggtactctgctggccctaattgctcttgttaaacctcggagcaaagtggtgttatgcggagaccccaagcaatgcggattcttcaatatgatgcagcttaaggtgaacttcaaccacaacatctgcactgaagtatgtcataaaagtatatccagacgttgcacgcgtccagtcacggccatcgtgtctacgttgcactacggaggcaagatgcgcacgaccaacccgtgcaacaaacccataatcatagacaccacaggacagaccaagcccaagccaggagacatcgtgttaacatgcttccgaggctgggcaaagcagctgcagttggactaccgtggacacgaagtcatgacagcagcagcatctcagggcctcacccgcaaaggggtatacgccgtaaggcagaaggtgaatgaaaatcccttgtatgcccctgcgtcggagcacgtgaatgtactgctgacgcgcactgaggataggctggtgtggaaaacgctggccggcgatccctggattaaggtcctatcaaacattccacagggtaactttacggccacattggaagaatggcaagaagaacacgacaaaataatgaaggtgattgaaggaccggctgcgcctgtggacgcgttccagaacaaagcgaacgtgtgttgggcgaaaagcctggtgcctgtcctggacactgccggaatcagattgacagcagaggagtggagcaccataattacagcatttaaggaggacagagcttactctccagtggtggccttgaatgaaatttgcaccaagtactatggagttgacctggacagtggcctgttttctgccccgaaggtgtccctgtattacgagaacaaccactgggataacagacctggtggaaggatgtatggattcaatgccgcaacagctgccaggctggaagctagacataccttcctgaaggggcagtggcatacgggcaagcaggcagttatcgcagaaagaaaaatccaaccgctttctgtgctggacaatgtaattcctatcaaccgcaggctgccgcacgccctggtggctgagtacaagacggttaaaggcagtagggttgagtggctggtcaataaagtaagagggtaccacgtcctgctggtgagtgagtacaacctggctttgcctcgacgcgacgtcacttggttgtcaccgctgaatgtcacaggcgccgataggtgctacgacctaagtttaggactgccggctgacgccggcaggttcgacttggtctttgtgaacattcacacggaattcagaatccaccactaccagcagtgtgtcgaccacgccatgaagctgcagatgcttgggggagatgcgctacgactgctaaaacccggcggcatcttgatgagagcttacggatacgccgataaaatcagcgaagccgttgtttcctccttaagcagaaagttctcgtctgcaagagtgttgcgcccggattgtgtcaccagcaatacagaagtgttcttgctgttctccaactttgacaacggaaagagaccctctacgctacaccagatgaataccaagctgagtgccgtgtatgccggagaagccatgcacacggccgggtgtgcaccatcctacagagttaagagagcagacatagccacgtgcacagaagcggctgtggttaacgcagctaacgcccgtggaactgtaggggatggcgtatgcagggccgtggcgaagaaatggccgtcagcctttaagggagcagcaacaccagtgggcacaattaaaacagtcatgtgcggctcgtaccccgtcatccacgctgtagcgcctaatttctctgccacgactgaagcggaaggggaccgcgaattggccgctgtctaccgggcagtggccgccgaagtaaacagactgtcactgagcagcgtagccatcccgctgctgtccacaggagtgttcagcggcggaagagataggctgcagcaatccctcaaccatctattcacagcaatggacgccacggacgctgacgtgaccatctactgcagagacaaaagttgggagaagaaaatccaggaagccattgacatgaggacggctgtggagttgctcaatgatgacgtggagctgaccacagacttggtgagagtgcacccggacagcagcctggtgggtcgtaagggctacagtaccactgacgggtcgctgtactcgtactttgaaggtacgaaattcaaccaggctgctattgatatggcagagatactgacgttgtggcccagactgcaagaggcaaacgaacagatatgcctatacgcgctgggcgaaacaatggacaacatcagatccaaatgtccggtgaacgattccgattcatcaacacctcccaggacagtgccctgcctgtgccgctacgcaatgacagcagaacggatcgcccgccttaggtcacaccaagttaaaagcatggtggtttgctcatcttttcccctcccgaaataccatgtagatggggtgcagaaggtaaagtgcgagaaggttctcctgttcgacccgacggtaccttcagtggttagtccgcggaagtatgccgcatctacgacggaccactcagatcggtcgttacgagggtttgacttggactggaccaccgactcgtcttccactgccagcgataccatgtcgctacccagtttgcagtcgtgtgacatcgactcgatctacgagccaatggctcccatagtagtgacggctgacgtacaccctgaacccgcaggcatcgcggacctggcggcagatgtgcaccctgaacccgcagaccatgtggacctggagaacccgattcctccaccgcgcccgaagagagctgcataccttgcctcccgcgcggcggagcgaccggtgccggcgccgagaaagccgacgcctgccccaaggactgcgtttaggaacaagctgcctttgacgttcggcgactttgacgagcacgaggtcgatgcgttggcctccgggattactttcggagacttcgacgacgtcctgcgactaggccgcgcgggtgcatatattttctcctcggacactggcagcggacatttacaacaaaaatccgttaggcagcacaatctccagtgcgcacaactggatgcggtccaggaggagaaaatgtacccgccaaaattggatactgagagggagaagctgttgctgctgaaaatgcagatgcacccatcggaggctaataagagtcgataccagtctcgcaaagtggagaacatgaaagccacggtggtggacaggctcacatcgggggccagattgtacacgggagcggacgtaggccgcataccaacatacgcggttcggtacccccgccccgtgtactcccctaccgtgatcgaaagattctcaagccccgatgtagcaatcgcagcgtgcaacgaatacctatccagaaattacccaacagtggcgtcgtaccagataacagatgaatacgacgcatacttggacatggttgacgggtcggatagttgcttggacagagcgacattctgcccggcgaagctccggtgctacccgaaacatcatgcgtaccaccagccgactgtacgcagtgccgtcccgtcaccctttcagaacacactacagaacgtgctagcggctgccaccaagagaaactgcaacgtcacgcaaatgcgagaactacccaccatggactcggcagtgttcaacgtggagtgcttcaagcgctatgcctgctccggagaatattgggaagaatatgctaaacaacctatccggataaccactgagaacatcactacctatgtgaccaaattgaaaggcccgaaagctgctgccttgttcgctaagacccacaacttggttccgctgcaggaggttcccatggacagattcacggtcgacatgaaacgagatgtcaaagtcactccagggacgaaacacacagaggaaagacccaaagtccaggtaattcaagcagcggagccattggcgaccgcttacctgtgcggcatccacagggaattagtaaggagactaaatgctgtgttacgccctaacgtgcacacattgtttgatatgtcggccgaagactttgacgcgatcatcgcctctcacttccacccaggagacccggttctagagacggacattgcatcattcgacaaaagccaggacgactccttggctcttacaggtttaatgatcctcgaagatctaggggtggatcagtacctgctggacttgatcgaggcagcctttggggaaatatccagctgtcacctaccaactggcacgcgcttcaagttcggagctatgatgaaatcgggcatgtttctgactttgtttattaacactgttttgaacatcaccatagcaagcagggtactggagcagagactcactgactccgcctgtgcggccttcatcggcgacgacaacatcgttcacggagtgatctccgacaagctgatggcggagaggtgcgcgtcgtgggtcaacatggaggtgaagatcattgacgctgtcatgggcgaaaaacccccatatttttgtgggggattcatagtttttgacagcgtcacacagaccgcctgccgtgtttcagacccacttaagcgcctgttcaagttgggtaagccgctaacagctgaagacaagcaggacgaagacaggcgacgagcactgagtgacgaggttagcaagtggttccggacaggcttgggggccgaactggaggtggcactaacatctaggtatgaggtagagggctgcaaaagtatcctcatagccatggccaccttggcgagggacattaaggcgtttaagaaattgagaggacctgttatacacctctacggcggtcctagattggtgcgttaatacacagaattctgattggatcccggtccgaagcgcgctttcccgggaactcgagttcactagtcgatcccgcggccgctttcgaacctaggcaagcatgcgggcccagtgggtaattaattgaattacatccctacgcaaacgttttacggccgccggtggcgcccgcgcccggcggcccgtccttggccgttgcaggccactccggtggctcccgtcgtccccgacttccaggcccagcagatgcagcaactcatcagcgccgtaaatgcgctgacaatgagacagaacgcaattgctcctgctaggagcttaattcgacgaataattggatttttattttattttgcaattggtttttaatatttccaaaaaaaaaaaaaaaaaaaaaaaaaaaaaaaaaaaaaaaaaaaaaaaaaaaaaaaaaaaaaaaaaaaaaactagaaatcgcgatttctagtctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcgcgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttctgtctaagcggatgccgggagcagacaagcccgtcagggcgcgtcagcgggtgttggcgggtgtcggggctggcttaactatgcggcatcagagcagattgtactgagagtgcaccatatcgacgctctcccttatgcgactcctgcattaggaagcagcccagtactaggttgaggccgttgagcaccgccgccgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatctggctagcgatgaccctgctgattggttcgctgaccatttccggggtgcggaacggcgttaccagaaactcagaaggttcgtccaaccaaaccgactctgacggcagtttacgagagagatgatagggtctgcttcagtaagccagatgctacacaattaggcttgtacatattgtcgttagaacgcggctacaattaatacataaccttatgtatcatacacatacgatttacctcacactata

(19) United States
(12) Patent Application Publication
(10) Pub. No.: US 2008/0070305 A1
(43) Pub. Date: Mar. 20, 2008

(54) INHIBITION OF EXPRESSION OF A TARGET GENE

(76) Inventors: Ulrich Certa, Allschwil (CH); Kenneth Lundstrom, Oberwil (CH)

Correspondence Address: HOFFMANN-LA ROCHE INC.;PATENT LAW DEPARTMENT, 340 KINGSLAND STREET, NUTLEY, NJ, 07110, US

(21) Appl. No.: 11/941,224

(22) Filed: Nov. 16, 2007

Related U.S. Application Data
(63) Continuation of application No. 09/994,412, filed on Nov. 27, 2001.

(30) Foreign Application Priority Data
Nov. 29, 2000 (EP) 00126113.0

(51) Int. Cl. C07H 21/04; C12N 15/00

(52) U.S. Cl. 435/455; 536/23.1

(57) ABSTRACT

The present invention relates to a process to inhibit the expression of a target gene in cells or tissues by infecting said cells or tissues with viral particles.

### FIELD OF THE INVENTION

The present invention relates to a process for inhibiting the expression of a target gene in eukaryotic cells or tissues. It also includes a cell wherein the inhibition of the expression of a target gene is specific and finally it concerns a kit comprising reagents for inhibiting transcription of a target gene in a cell.

### BACKGROUND OF THE INVENTION

The specific inhibition of gene expression has a huge impact on therapeutic research. More precisely, it would be useful to develop a technique to specifically inhibit the function of individual genes.

In particular, it would be useful to prevent the progression of specific diseases, like cancers, infectious diseases or neurological disorders by inhibiting the function of specific genes, for example.

It would also be useful to be able to analyze the differences between normal and diseased tissues.

Furthermore, it would be of advantage for the study of cell proliferation, for the analysis of gene function or the functional alteration of gene expression. Certain genes may be required for cell or organism viability at only particular stages of the development.

Classical genetic techniques have been used to characterize mutations in organisms with reduced expression of selected genes. Such techniques require laborious screening programs and have been limited to organisms in which genetic manipulation has been already established.

These difficulties may be overcome by a method of using double stranded (ds) RNA interference to inhibit gene expression in mammalian cells.

The technique is based on the delivery of ds RNAs into cells, where interference with specific messenger RNA (mRNA) molecules will occur to inhibit gene expression.

In the International Patent Application WO 99/32619, a method to inhibit specifically gene expression in an invertebrate model organism is described. This method is based on the use of ds RNAs and their introduction into a living cell to inhibit gene expression of a target gene in that cell. The ds RNAs are introduced into the cell, i.e. intracellularly, or extracellularly, i.e. within a body cavity.

In the international patent application WO 99/32619, the use of a viral construct packaged into a viral particle may be efficient for introduction of an expression construct into the cell and the transcription of RNA encoded by the expression construct.

Constructs with both sense and anti-sense sequences in the same viral vector did not successfully inhibit gene expression, most likely due to inefficient interaction with target mRNA. It was postulated that when the sense and the anti-sense RNAs are encoded by one construct, the RNA duplex formation occurs immediately and no interaction with mRNAs is possible.

More recently, in a scientific publication (F. Wianny and M. Zernicka-Goetz, Specific interference with gene function by double stranded RNA in early mouse development, Nature Cell Biology, vol. 2, February 2000, pp. 70-75), it is described that synthetic ds RNAs have been introduced into both mouse oocytes and preimplantation of embryos carried out by microinjection and specific inhibition of gene expression was achieved.

One major difficulty is, at present, the delivery of the ds RNA into cells efficiently. No genetic technique in this domain has been developed for direct introduction of ds RNAs into cells.

Clearly, the possibility to introduce ds RNAs biologically and not mechanically into cells would be beneficial. Such introduction reduces the manipulations and circumvents the generation of mechanical cell damage.

Furthermore, the ability to inhibit a specific target gene without affecting other genes of the cell would be of great importance.

Finally, the ability to inhibit a specific target gene at a specific time and at a defined location in tissues or organisms without introduction of permanent mutations into the target genome would be of substantial interest.

### SUMMARY OF THE INVENTION

The present invention provides a process to inhibit the expression of a target gene in cells or tissues comprising infection of said cells or tissues with (a) viral particles containing single stranded ribonucleic acid (ss RNA) expressing a sense RNA strand and (b) viral particles containing single stranded ribonucleic acid (ss RNA) in anti-sense RNA strand, wherein the sense and anti-sense RNA strands comprise homologous nucleotide sequences to a portion of said target gene. The present invention relates also to a cell wherein two complementary RNA strands interfere with the expression of a target gene, it concerns a kit comprising reagents for inhibiting transcription of a target gene in cells or tissues and finally the use of the claimed process for the treatment and the prevention of disease abd a pharmaceutical composition.

### DETAILED DESCRIPTION OF THE INVENTION

The expression “ds RNA” as used herein means double stranded RNA.

The expression “ss RNA” as used herein means single stranded RNA.

The term “sense” as used herein means a RNA sequence corresponding to strand of the mRNA

The term “anti-sense” as used herein means a RNA complimentary sequence to the sense strand of the mRNA.

The expression “sequence specific for” as used herein means that the sequence of the sense and anti-sense RNA strands has at least 90%, preferably 95%, more preferably 90% and most preferably 100%, bases identically to the said target gene.

The process of the present invention for inhibiting the expression of a target gene in cells or tissues comprises infection of said cells or tissues with (a) viral particles containing single stranded ribonucleic acid (ss RNA) expressing a sense RNA strand and (b) viral particles containing single stranded ribonucleic acid (ss RNA) in anti-sense RNA strand, wherein the sense and anti-sense RNA strands comprise homologous nucleotide sequences to a portion of said target gene.

The present invention is useful for selective inhibition of specific gene functions by biologic generation of ds RNAs in the cells in contrast to mechanical introduction of ds RNAs into the cells or the tissues. In particular, it would be useful for the treatment or the prevention of specific diseases or pathologies to inhibit specific over-expression of genes, which is required for the initiation or the maintenance of said diseases or said pathologies. Treatment would include amelioration of any symptoms associated with the disease or clinical indications associated with the pathology.

For example, the present invention may be useful for treatment or prevention of patients suffering from tumors by inhibition of specific gene function. Tumors include ovary, prostate, breast, colon, liver, stomach, brain, head-and-neck and lung cancers.

Another use of the present invention could be a method to identify gene function in an organism by specific inhibition of expression.

Furthermore, the present invention may be useful for analysis and prevention of the mechanism for growth, development, metabolism, disease resistance or other biological processes.

The advantage of the present invention include: the ease of biological generation of ds RNAs into cells or tissues, the highly efficient amplification of the introduced ss RNAs, the stability of ds RNAs in cells and tissues and the efficiency of the inhibition and the biological safety.

The term “alphavirus” has its conventional meaning in the art, and includes the various species of alphaviruses such as Eastern Equine Encephalitis virus (EEE), Venezuelan Equine Encephalitis virus (VEE), Everglades virus, Mucambo virus, Pixuna virus, Western Equine Encephalitis virus (WEE), Sindbis virus, South African Arbovirus No. 86, Semliki Forest virus, Middelburg virus, Chikungunya virus, O'nyong-nyong virus, Ross River virus, Barmah Forest virus, Getah virus, Sagiyama virus, Bebaru virus, Mayaro virus, Una virus, Aura virus, Whataroa virus, Babanki virus, Kyzylagach virus, Highlands J virus, Fort Morgan virus, Ndumu virus, and Buggy Creek virus. The term “also includes vectors derived thereof. alphavirus” Te preferred alphavirus include Semliki Forest Virus (SFV) (Liljeström and Garoff, 1991). A new generation of animal cell expression vectors based on the Semliki Forest virus replicon, Bio/Technology 9, 1356-1361), Sindbis Virus (SIN) (Xiong et al., 1989 Sindbis virus: an efficient broad host range vector for gene expression in animal cells, Science 243, 1188-1191) and Venezuelan Equine Encephalitis Virus (VEE) (Davis et al., 1989 In vitro synthesis of infectious Venezuelan equine encephalitis virus RNA from a cDNA clone: analysis of a viable deletion mutant, Virology 171, 189-204), for example. The alphavirus and vectors are well-known in the art and commercially available.

In the process of the present invention, said cells or tissues are infected with an amount of viral particles containing ss RNA, which allows delivery of at least one copy per cell. As disclosed herein, the infection is made with a number superior or equal to 10 viral particles per cell.

The infection procedure is well known in the art. The in vitro infection in cell lines and primary cell cultures, like fibroblasts, hepatocytes, neurons, for example, is carried out by addition of SFV particles directly to the cell cultures. The viral particles will recognize receptors on the cell surface, penetrate the cell membrane either by fusion or endocytosis (depending on cell type), where after the RNA molecules will be liberated into the cytoplasm (“The Alphaviruses: Gene Expression, Replication, and Evolution, Strauss”, J. H and Strauss, E. G., 1994, Microbiological Reviews 58, 491-562).

The in vivo infection requires injection of the SFV particles to the target tissue. Injection of SFV particles (“Efficient in vivo expression of a reporter gene in rat brain after injection of recombinant replication-deficient Semliki Forest virus”, Lundstrom, K., Grayson, J. R., Pink J. R. and Jenck, F., 1999, Gene Therapy & Molecular Biology 3, 15-23) will result in a similar infection procedure as described for the in vitro situation above.

Cells or tissues in the present process are infected with separate viral particles expressing complementary strands, sense and anti-sense RNA strands.

As disclosed herein, cells or tissues in the present process may be co-infected with equal amounts of viral particles containing sense RNA strand and of viral particles containing anti-sense RNA strand, respectively, to allow the formation of ds RNAs capable of interfering with gene expression. Higher doses of ds RNA may yield more effective inhibition.

In the present invention the viral particles contains the ss RNA strand comprising homologous nucleotide sequences to a portion of the said target gene and this ss RNA strand is cloned into the vector of the alphavirus. The ss RNA strand may be cloned either in sense or anti-sense orientation into the said vector. The other genes present in the vector are the nonstructural alphavirus genes especially the nsP-1-4 genes (The Alphaviruses: Gene Expression, Replication, and Evolution, Strauss, J. H and Strauss, E. G., 1994, Microbiological Reviews 58, 491-562), responsible for RNA replication in host cells. Expression of nsP1-4 results in the formation of the replicase complex, that will initiate extensive RNA replication, i.e. generation of large numbers of sense and anti-sense RNA capable of efficient ds RNA formation.

The process herein, allows in general, inhibition of many different types of target genes in eukaryotic cells or tissues. The target gene may be a eukaryotic gene, a viral gene, a gene of a pathogen or a synthetic gene. Clearly, the target gene may be a gene derived from the cell, i.e. a cellular gene, a transgene, i.e. a gene construct inserted at an ectopic site in the genome of the cell or a gene from a pathogen, capable of infecting an organism from which the cell is derived.

The target genes may be any gene of interest, there already having been a large number of proteins of interest identified and isolated. The target gene may be a developmental gene, like cyclin kinase inhibitors, growth/differentiation factors and their receptors, telomerase reverse transcriptase (TERT), an oncogene, a tumor suppressor gene or an enzyme, for example. A gene derived from any pathogen may be the target of inhibition.

Since inhibition in the present invention is sequence specific, sense and anti- sense RNA strands introduced into the cells or tissues comprise a complementary nucleotide sequence of a portion of the target gene.

A complete homology between the RNA and the target gene is not required to practice the present invention. As disclosed herein, sequence variations due to genetic mutations, polymorphisms, or evolutionary divergences, for example, are tolerated. RNA strands with insertions, deletions, and single point mutations to the target gene have been found to be effective for inhibitions.

The length of the said homologous nucleotide sequence should be at least 50 bases, preferably 75, 100 or 125 bases.

In the process of the present invention, the inhibition of the target gene expression demonstrates a loss of phenotype. Depending on the target gene and the intracellular dose of ds RNA, the process of the present invention may result in partial or complete loss of function of the target gene in the cells or tissues of the organism.

Inhibition of gene expression refers to the absence or the decrease in the level of protein and/or mRNA from a target gene. The consequences of inhibition may be assayed for properties of the cell or organism by molecular biology methods such as RNA solution hybridization, Northern hybridization (Sambrook et al., Molecular Cloning, vol. 1, 7.37 & 7.39) and biochemical assays like enzyme linked immunoabsorbent assay (ELISA), Western blotting (Towbin et al. 1979; Bunette 1981 or Sambrook et al., Molecular Cloning, vol. 3, 18.60) or radioimmunoassay (RIA) (Sambrook et al., Molecular Cloning, vol. 3, 18.19-18.20), for example.

The degree of inhibition may be estimated by comparing the values from untreated cells to those obtained from cells treated according to the method of the present invention.

The present invention concerns also any cell containing two complementary RNA strands, a sense and an anti-sense RNA strand, which form a double stranded RNA inside the said cell and because of nucleotide sequence homology to a portion of a specific target gene are capable of interfering with the expression of the said target gene.

As disclosed herein, the eukaryotic cells or tissues with the target gene may be any cell or tissue type, which can be infected by an alphavirus.

They may be from the vascular or extravascular circulation, from the blood or lymph system, from muscles, liver, brain, or from the cerebrospinal fluid, for example.

The eukaryotic cells or tissues may be contained in any organism including fish, amphibians, reptilians, insects or mammal like cattle, pig, hamster, mouse, rat, primate and human, for example.

Furthermore, the present invention claims a kit comprising reagents to inhibit expression of a target gene, wherein said kit comprises of at least a sufficient amount of single stranded RNA viral particles expressing either sense or anti-sense RNA strand, which are complementary to each other and form a ds RNA comprising a homologous nucleotide sequences to a portion of said target gene and capable to interfere with the expression of the said target.

Such a kit may include reagents necessary to carry out the in vivo or in vitro delivery of RNA to test samples or subjects.

Such a kit may also include instructions to allow a user of the kit to practice the invention.

The use of the process of the present invention is also claimed for treatment or prevention of disease.

To treat a disease or pathologic condition, a target gene may be selected which is expressed during the development of the disease or which is the cause of the pathologic condition.

To prevent a disease or a pathologic condition, a target gene may be selected which is required for initiation and/or maintenance of the disease or the pathologic condition.

The present invention may be used for treatment or prevention of cancer including solid tumors or leukemias, by co-infection of tumors with viral vectors carrying sense and anti-sense RNA with the aim of generating ds RNA for the inhibition of mRNA translation of a gene required for the maintenance of the carcinogenic/tumorigenic phenotype.

The present invention may be used for treatment or prevention of infectious diseases due to a pathogen, for example. Cells or tissues infected or which may be infected by human immunodeficiency virus (HIV) may be targeted according to the present invention in order to inhibit the expression of a specific gene responsible or required for initiation and/or maintenance of said infection.

The present invention concerns also the use of (a) viral particles containing single stranded ribonucleic acid (ss RNA) expressing sense RNA strand and (b) viral particles containing single stranded ribonucleic acid (ss RNA) expressing anti-sense RNA strand, wherein the sense and anti-sense RNA strands comprise homologous nucleotide sequences of a portion of a target gene for the preparation of a medicament for treating diseases.

Finally the present invention concerns a pharmaceutical composition comprising (a) viral particles containing single stranded ribonucleic acid (ss RNA) expressing sense RNA strand and (b) viral particles containing single stranded ribonucleic acid (ss RNA) expressing anti-sense RNA strand, wherein the sense and anti-sense RNA strands have homologous nucleotide sequences of a portion of a target gene and optionally pharmaceutically acceptable excipients for the inhibition of the expression of the said target gene in cells or tissues.

The present invention will be better understood on the basis of the following examples, offered by way of illustration and not by way of limitation.

The examples below are in connection with the following figures:

### FIGURES

FIG. 1: Inhibition of Aldolase A expression with block stocks.

Panel A: Schematic representation of the human aldolase A gene and probes used for expression analysis. A and B are fragments used to probe Northern blots (FIG. 2). V and G are primers pairs that amplify either RNA expressed by the virals stocks or else selectively chromosomal transcripts.

Panel B: Detection of either virally encoded aldolase mRNA with VA/VB (first two pictures form top) or endogenously encoded mRNA using (GA/GB lower panels). Co are unifected control cells, s is infected with the sense strand virus and as with the antisense virus and ds represents a 1:1 mixture of both virus stocks (block stock).

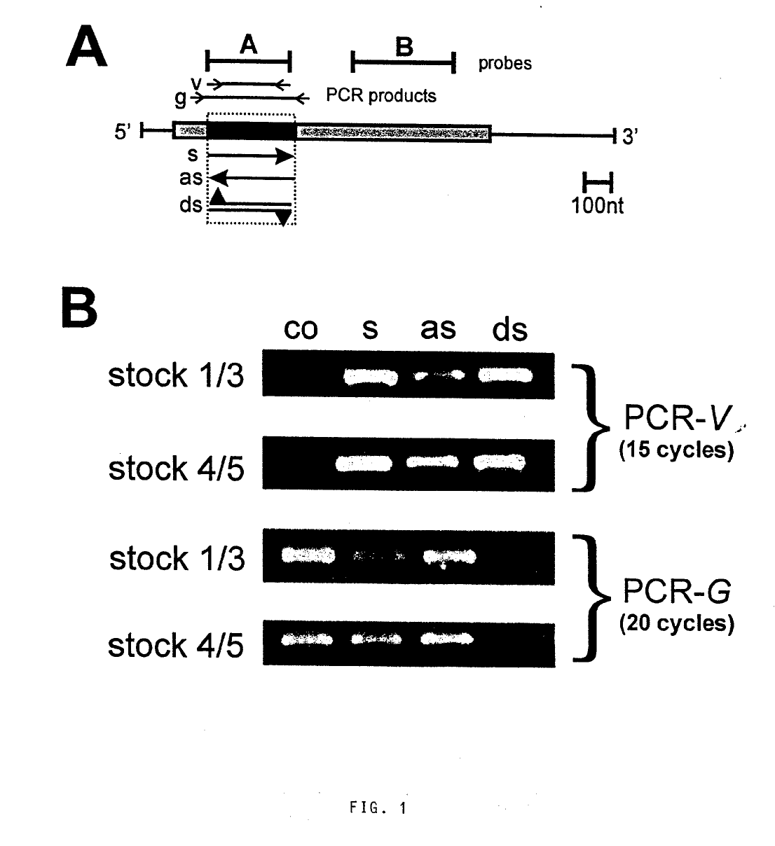

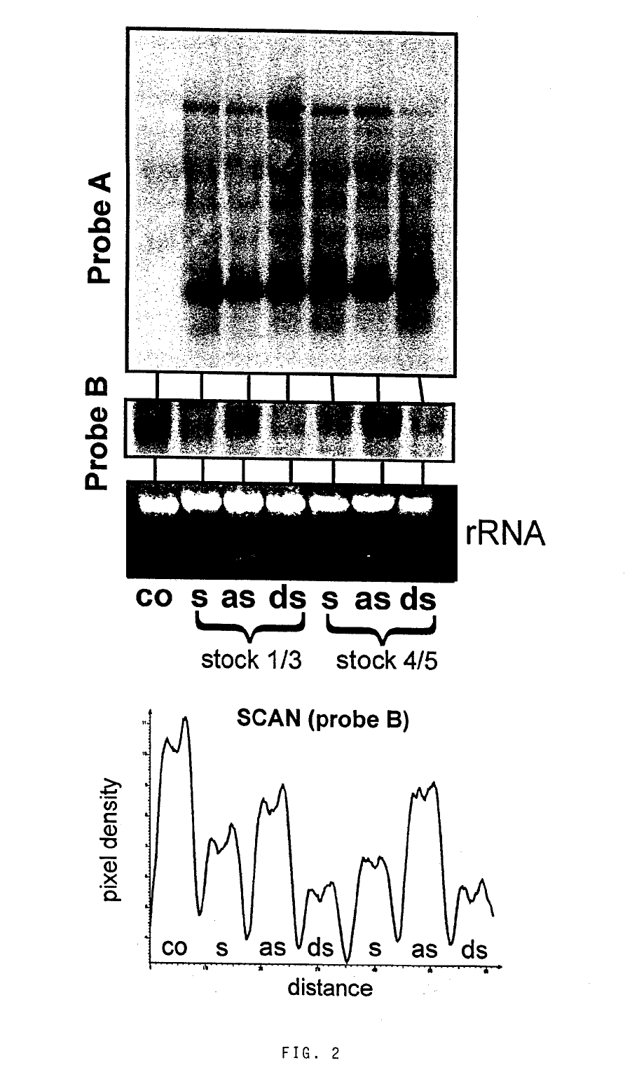

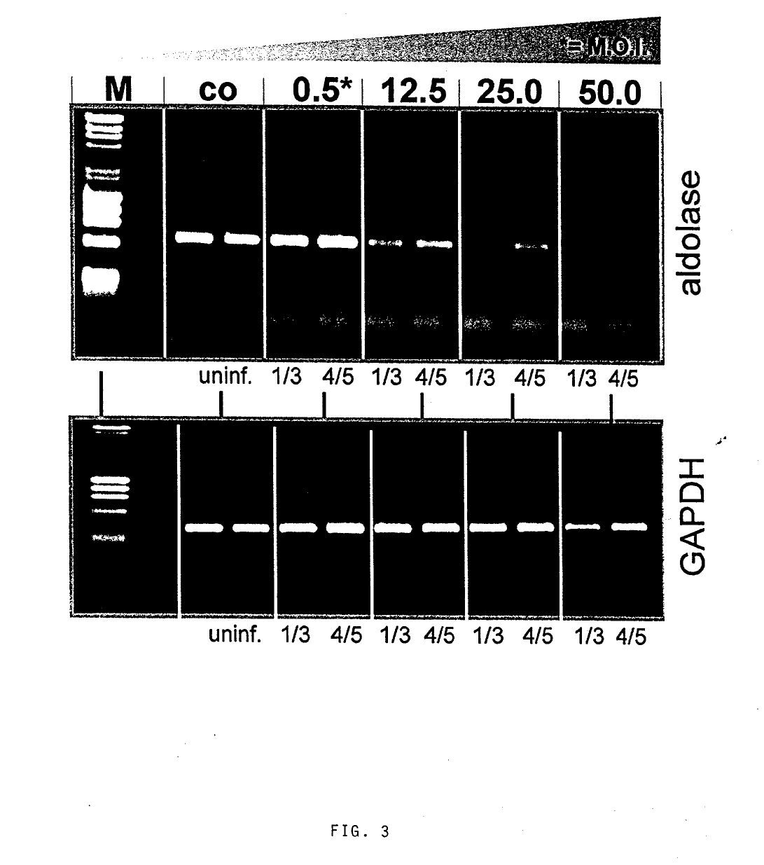

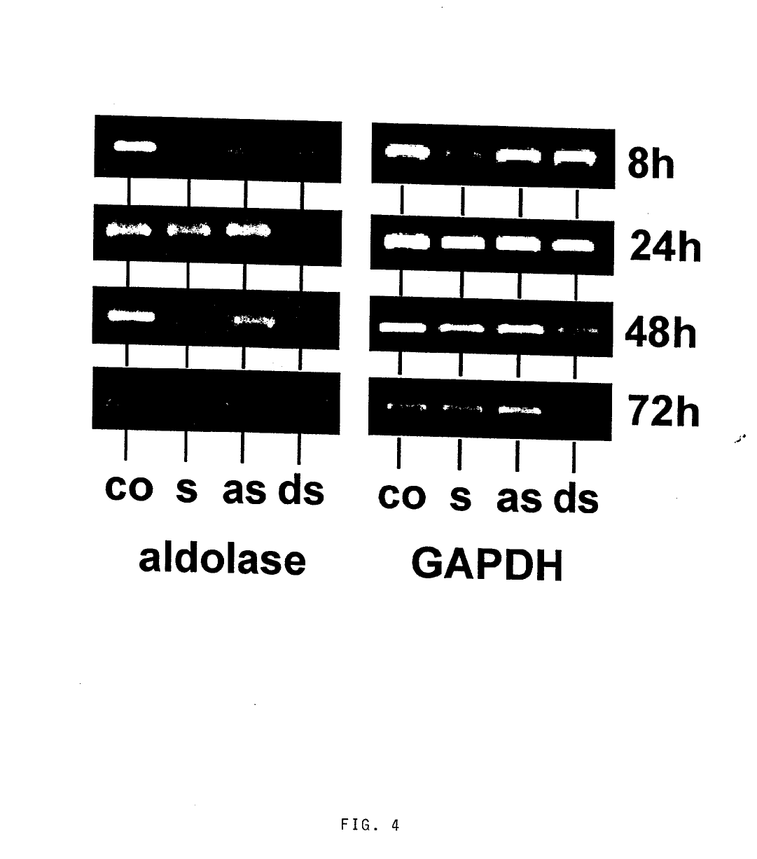

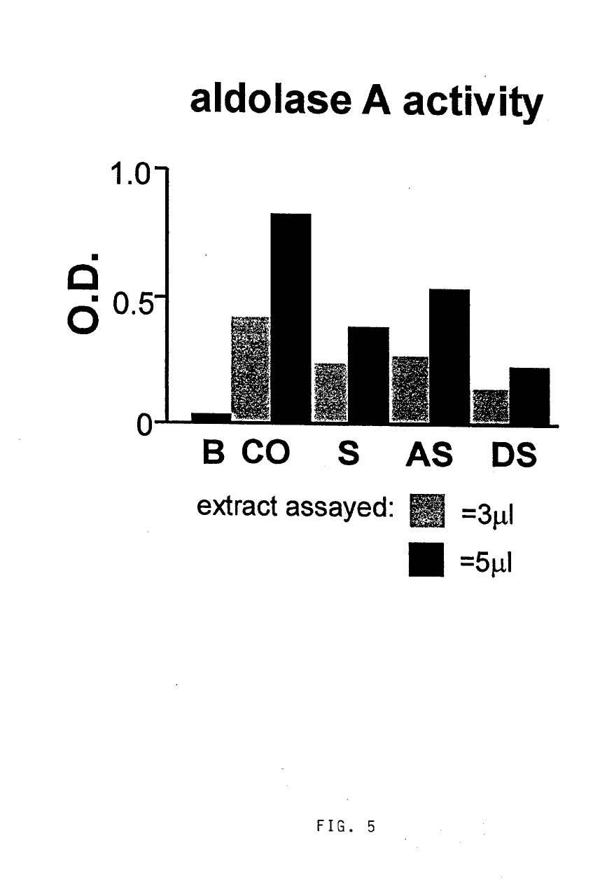

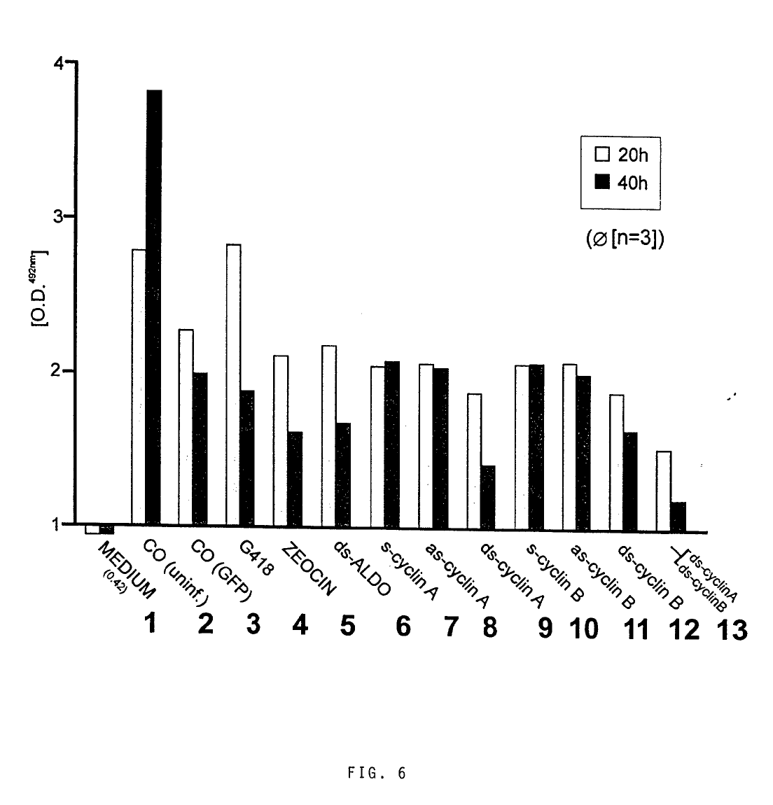

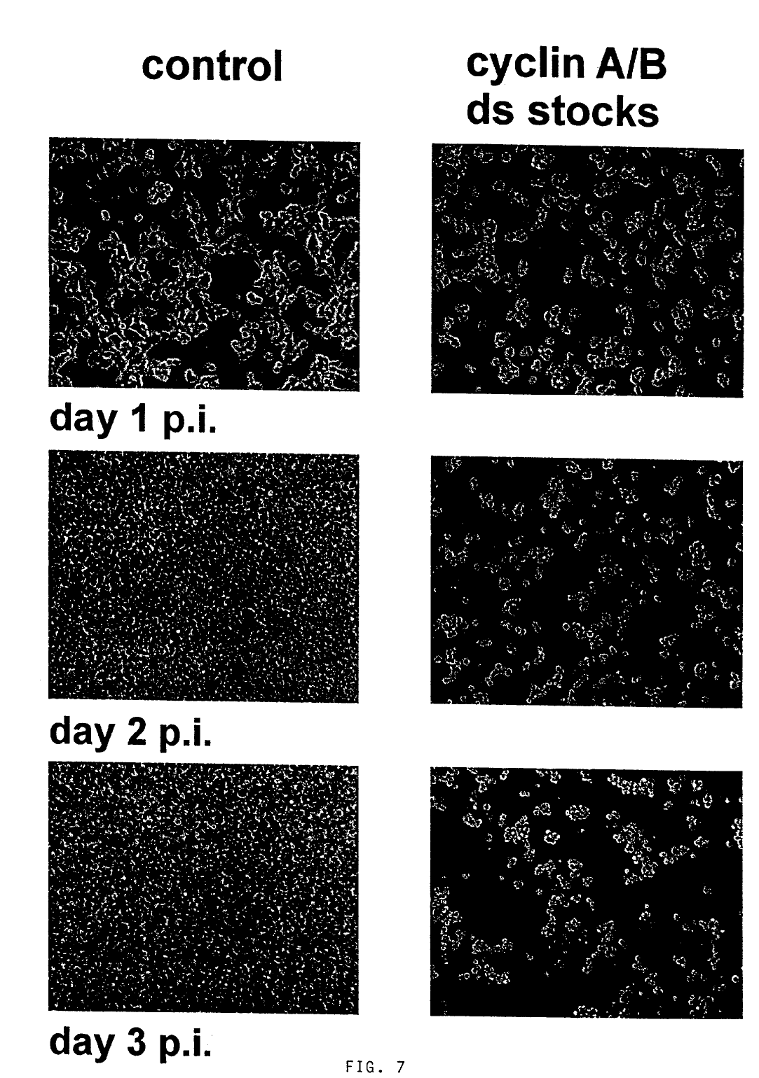

FIG. 2: Analysis of aldolase RNA by Northern blots. Top panel: Total RNA of infected cells (see FIG. 1) was separated by gel analysis transferred to a membrane and probed either with labeled A which covers the virally expressed region or with B that is specific for chromosomal copies of aldolase A RNA. Probe A required 1.5 h of exposure and B overnight exposure to x-ray film. At the bottom is shown a scan of the autoradiograph of the probe B blot.

FIG. 3: Correlation between inhibition of gene transcription and virus titration.

Cells were infected with a M.O.I. (multiplicity of infection) of 0.5 to 50 with s/as virus block stocks and incubated for 24 hours. Total RNA was isolated and converted into cDNA. PCR was carried out for 25 cycles either with aldolase or GAPDH (control) specific primers. The amplicons were visualized by conventional agarose electrophoresis. The results with two independent virus block stocks are shown (1/3 and 4/5).

FIG. 4: Kinetics of inhibition by as/s virus stocks. HEK (Human embryonic kidney) cells were infected with 1/3 block stocks or the individual s and as stocks. The cells were incubated for the times indicated followed by PCR analysis of the transcript levels.

FIG. 5: Measurement of aldolase A enzyme activity.

Co indicates the enzyme level in unifected cells and B is a buffer, negative control, as, s and block stocks (ds) were used to infect the cells at MOI of 25 for 24 hours. Cells were harvested lysed and the enzyme activity was measured using a commercial assay and either 3 or 5 μl lysate.

FIG. 6: Cell cycle arrest by cyclin down-regulation.

From left to right: 1. medium control; 2. uninfected cells (maximal proliferation); 3. cells infected with a virus expressing green fluorescent protein (GFP, infection control); 4. and 5. assay control with antibiotics G418 and zeocin; 6. human aldolase A dsRNA (inhibition control); 7. cyclin A sense SFV; 8. cyclin A antisense SFV; 9. cyclin A sense and antisense SFV (ds); 10. cyclin B sense SFV; 11. cyclin B antisense SFV; 12. cyclin B sense and antisense SFV (ds); 13. cyclin A and cyclin B sense and antisense SFV (ds).

FIG. 7: Microscopic image of culture of cells infected with virus expressing GFP and culture of cyclin A and cyclin B sense and antisense SFV.

In the examples below the methods and techniques required are known from the literature and are described, for example, in Sambrook et al., 1989.

In the examples below, the SFV vector used is a noncytopathogenic version with two point mutations in the SFV nonstructural gene nsP2 (Ser259Pro and Arg650Asp) described by Lundstrom, K., Schweitzer, C., Richards, J. G., Ehrengruber, M. U., Jenck, F. and Muelhardt, C. 1999, Semliki Forest virus vectors for in vitro and in vivo applications. This modified SFV vector do not inhibit the endogenous gene expression in the infected host cells, which allows targeted and specific gene inhibition by the dsRNA technology.

### EXAMPLES

### Example 1

### Inhibition of Aldolase A Expression in BHK (Baby Hamster Kidney) Cells (ATCC Registered Number: CCL-10) (FIG. 1.)

Based on the human aldolase A gene (M. Sakakibara, T. Mukai & K. Hori, Nucleotide sequence of a cDNA clone for human aldolase: a messenger RNA in the liver, Biochem. Biophys. Res. Commun. 30, 413-420, 1985) 3 pairs of oligonucleotide primers were selected to amplify the required gene regions. VA (nt 210-240 as described by M. Sakakibara, T. Mukai & K. Hori, Nucleotide sequence of a cDNA clone for human aldolase: a messenger RNA in the liver, Biochem. Biophys. Res. Commun. 30, 413-420, 1985) and VB (nt 740-710 as described by M. Sakakibara, T. Mukai & K. Hori, Nucleotide sequence of a cDNA clone for human aldolase: a messenger RNA in the liver, Biochem. Biophys. Res. Commun. 30, 413-420, 1985) amplify a region of about 600 nucleotides used for construction of the sense and antisense virus stocks. GA (nt 170-200 as described by M. Sakakibara, T. Mukai & K. Hori, Nucleotide sequence of a cDNA clone for human aldolase: a messenger RNA in the liver, Biochem. Biophys. Res. Commun. 30, 413-420, 1985) and GB (nt 780-750 as described by M. Sakakibara, T. Mukai & K. Hori, Nucleotide sequence of a cDNA clone for human aldolase: a messenger RNA in the liver, Biochem. Biophys. Res. Commun. 30, 413-420, 1985) amplify a chromosomal region of the aldolase gene. Northern Probe A is generated using primers VA and VB and probe B was amplified with a primer pair of the upstream region (nt 951-980 and nt 1330-1301 as described by M. Sakakibara, T. Mukai & K. Hori, Nucleotide sequence of a cDNA clone for human aldolase: a messenger RNA in the liver, Biochem. Biophys. Res. Commun. 30, 413-420, 1985). Cells were infected and grown for 24 hours. RNA was isolated and converted into cDNA according to standard procedures. All PCR products were subcloned into common cloning vectors for sequencing. The VA/VB was further cloned into the SFV vector to generate infectious SFV particles. The virally encoded aldolase mRNA is abundant and detected after 15 cycles of PCR in virus infected cells. No signal is obtained in cells without virus. Using the genomic primers for aldolase mRNA a band of the expected size is amplified in the uninfected cells and cells infected with sense or antisense producing viruses. The mixture of both the sense and antisense viruses is a potent inhibitor of expression of the chromosomal aldolase gene whilst the viral gene expression remains unaffected.

### Example 2

### Analysis of Aldolase RNA by Northern Blots (FIG. 2)

Total RNA from either uninfected cells or cells infected with the virus stocks indicated was separated on a standard formamide gel, transferred after electrophoresis to a nitrocellulose membrane and then probed either with radiolabeled fragment A or B (see example 1). Probe A detects almost exclusively the virus-derived aldolase RNA due to the short exposure time of 45 minutes. Probe B detects only chromosomally encoded aldolase mRNA after 16 hours exposure of the hybrized blot to film. A stained gel with ribosomal RNA was used to control loading (below probe B). Especially in the gel scan, it is evident that the aldolase mRNA levels are lowest in the cells infected with both viruses.

### Example 3

### The Inhibition of Gene Transcription is Dependent on Virus Titers

As it can be seen in FIG. 3, relative to the control, the levels of aldolase mRNA start to decrease at a M.O.I. of 12.5 and at 50 essentially no mRNA can be detected using this sensitive assay. The levels of another chromosomal control gene (GAPDH) are not altered with increasing M.O.I.

### Example 4

### Kinetics of Inhibition by as/s Virus Stocks

BHK cells (ATCC registered number: CCL-10) were infected with as or s or an as/s mix of aldolase RNA virus stocks. At the time points indicated in the figure, RNA was isolated and converted into cDNA. After PCR the products were analyzed by agarose gel electrophoresis. At 8 hours marginal destruction of genomic aldolase RNA is evident and the highest activity is detectable at 48 hours. In this particular experiment also the sense expressing virus influenced RNA stability. The GAPDH RNA remains unaltered except in the 48 and 72 h samples, in which a reduction of the RNA levels is evident in cells infected with the s/as virus mix. This is probably related to cell death because aldolase is an essential enzyme.

### Example 5

### Reduction of Aldolase Enzyme Activity by s/as Aldolase Virus Stocks

BHK cells (ATCC registered number: CCL-10) were infected with the stocks indicated and grown for 24 hours under standard cell culture conditions. The cells were harvested and lysed in 1×PBS containing 0.2% Triton X-100. After centrifugation for 10 min at 16′000 g and 4° C. the supernatant was recovered and either 3 μl (grey bars) or 5 μl (black bars) were assayed using a commercial kit (SIGMA, catalogue #: 752-A) and the protocol supplied. The most significant reduction of enzyme activity is as expected in the sample infected with both the s and as virus stocks.

### Example 6

### Cyclin “Knock Down” Results in Cell Cycle Arrest

Cell cycle arrest by cyclin down-regulation. Human embryonic kidney (HEK293) cells (ATCC registered number: CRL-1573) were infected with the SFV virus particles indicated at time point zero and proliferation was assayed after 20 (light grey bars) and 40 hours (dark grey bars) in culture using a commercial color assay (Promega G5421 according to technical bulletin TB245). The mixture of the cyclin A and B blocking virus stocks was most efficient and even more potent than inhibition of cell growth by antibiotics (neomycin and zeocin).

The sequence of the cyclin A is those described in (“Hepatitis B virus integration in a cyclin A gene in a hepatocellular carcinoma”, Wang, Chevenisse X., Henglein B., Brechot C., Nature 343:555-557(1990)) and the sequence of the cyclin B si those described by (Kim D. G., Choi S. S., Kang Y. S., Lee K. H., Kim U.-J., Shin H.-S., Submitted (06-MAY-1997) to the EMBL/GenBank/DDBJ databases, Life Science, Pohang University of Science and Technology, San 31, Pohang, Kyungbuk 790-784, Korea).

### Example 7

### Culturing of Cells Infected with Sense and Anti-Sense in one Vector

Sense and anti-sense fragments of the cyclin A and B genes were cloned into a single SFV vectors by the introduction of a second subgenomic 26S promoter. The constructs were the following:

SFV 26S—sense cyclin A—SFV 26S—anti-sense cyclin A and

SFV 26S—sense cyclin B—SFV 26S—anti-sense cyclin B

Infections of HEK293 cells with SFV-cyclin A or SFV-cyclin B alone, or together, did not result in any arrest of cell proliferation.

This indicated that constructs with both sense and anti-sense fragments in the same vector are not able to inhibit expression of chromosomal cyclin genes.

1. A process to inhibit the expression of a target gene in cells or tissues comprising infection of said cells or tissues with (a) viral particles containing single stranded ribonucleic acid (ss RNA) expressing a sense RNA strand and (b) viral particles containing single stranded ribonucleic acid (ss RNA) in anti-sense RNA strand, wherein the sense and anti-sense RNA strands comprises homologous nucleotide sequences to a portion of said target gene.

2. The process of claim 1 in which the viral particles are alphaviruses.

3. The process of claims 1 to 2 in which for infection the viral particles containing ss RNA expressing sense RNA strand are in equal amounts to those containing ss RNA expressing anti-sense RNA strand.

4. The process of claims 1 to 3 in which said single stranded RNA is cloned either in sense or anti-sense orientation into the vector of the said viral particle.

5. The process of claims 1 to 4 in which said target gene is an eukaryotic gene, a viral gene or a synthetic gene.

6. The process of claims 1 to 5 in which said homologous nucleotide sequence is specific for the said target gene and at least 50 bases in length.

7. The process of claims 1 to 6 in which the cells or tissues are present in an organism and inhibition of said target gene expression demonstrates a phenotypic loss-of-function.

8. A cell containing two complementary RNA strands, a sense and an anti-sense RNA strand, which form inside the said cell a double stranded RNA comprising an homologous nucleotide sequences to a portion of a specific target gene and capable to interfering with the transcription of the said target gene.

9. A kit comprising reagents to inhibit the expression of a target gene in cells or tissues, wherein said kit comprises at least a sufficient amount of single stranded RNA (ss RNA) viral particles expressing either sense or anti-sense RNA strand which are complementary and form inside said cells or tissues a ds RNA comprising an homologous nucleotide sequence to a portion of said target gene and capable to interfering with the expression of the said target.

10. Use of the process of claims 1 to 10 for treatment or prevention of disease.

11. Use of (a) viral particles containing single stranded ribonucleic acid (ss RNA) expressing sense RNA strand and (b) viral particles containing single stranded ribonucleic acid (ss RNA) expressing anti-sense RNA strand, wherein the sense and anti-sense RNA strands comprise homologous nucleotide sequences of a portion of a target gene for the preparation of a medicament for treating diseases.

12. A pharmaceutical composition comprising (a) viral particles containing single stranded ribonucleic acid (ss RNA) expressing sense RNA strand and (b) viral particles containing single stranded ribonucleic acid (ss RNA) expressing anti-sense RNA strand, wherein the sense and anti-sense RNA strands comprise homologous nucleotide sequences of a portion of a target gene and optionally pharmaceutically acceptable excipients for the inhibition of the expression of the said target gene in cells or tissues.
